(19) United States
(12) Patent Application Publication
(10) Pub. No.: US 2008/0119839 A1
(43) Pub. Date: May 22, 2008

(54) Cryosurgical Applicator

(76) Inventor: David W. Vancelette, San Diego, CA (US)

Correspondence Address: AMS RESEARCH CORPORATION, 10700 BREN ROAD WEST, MINNETONKA, MN, 55343, US

(21) Appl. No.: 11/943,711

(22) Filed: Nov. 21, 2007

Related U.S. Application Data
(60) Provisional application No. 60/866,690, filed on Nov. 21, 2006.

(51) Int. Cl. A61B 18/02

(52) U.S. Cl. 606/23

(57) ABSTRACT

A cordless cryosurgical applicator system having a base unit and one or more rechargeable cryosurgical applicators. The base unit can be use to charge the applicator with a cryogenic fluid such that a cryosurgical procedure can be performed. Following use of the applicator, the applicator can be recharged by attaching the applicator to the base unit and refilling a cryogenic reservoir in the applicator from a cryogenic storage tank contained within the base unit. The base unit can include a self-contained assembly for generating a cryogenic fluid such that the cordless cryosurgical applicator system is essentially self-sufficient when attached to an electrical power source.

### PRIORITY CLAIM

The present application claims priority to U.S. Provisional Application Ser. No. 60/866,690, filed Nov. 21, 2006 and entitled “CRYOSURGICAL APPLICATOR”, which is herein incorporated by reference in its entirety.

### FIELD OF THE INVENTION

The present disclosure relates to a tool used in performing cryosurgery and more particularly to a cryosurgical system providing compact and versatile, handheld cryosurgical tools that are recharged using a base unit that includes a self-contained, refrigerant recharging system.

### BACKGROUND OF THE INVENTION

Cryosurgery is used to treat a variety of diseases. In cryosurgery, diseased body tissue is quickly frozen, causing the tissue to die after which it will be absorbed by the body, expelled by the body, sloughed off or replaced by scar tissue. Cryosurgery can be used to treat prostate cancer and benign prostate disease. Cryosurgery also has gynecological applications. In addition, cryosurgery may be used for the treatment of a number of other diseases and conditions including, but certainly not limited to, breast cancer, liver cancer, renal cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

A representative system for performing cryosurgery is the Her Option® Office Cryoablation Therapy available from American Medical Systems of Minnetonka, Minn. In such a system one or more fluids are stored in a control console and are circulated through a cryoprobe used to perform the cryosurgical procedure. Typically, the fluid is transferred between the console and the cryoprobe through a non-detachable flexible line connecting the console and the cryoprobe. In some instances, this flexible connection can be less than optimal due to inherent limitations of reach and maneuverability of the cryoprobe. The flexible line can also create a weak point in the circuit, which is vulnerable to perforation of the channels through which fluid flows and loss of system functionality.

One representative cryosurgical system that has been developed without flexible lines is the CryoPen available from CryoPen LLC and disclosed in U.S. Pat. Nos. 6,430,956 and 6,629,417. The CryoPen utilizes a handheld thermal mass having an exposed tip, wherein the handheld thermal mass and tip are cooled for use through interfacing the handheld thermal mass with a heat exchange system. However, the CryoPen has limited cooling capacity due to the cooling being solely provided by the thermal mass acting as a heat sink.

As such, it would be advantageous to further improve on existing cryosurgical systems by improving the maneuverability and safety of a cryoprobe while maintaining an effective amount of cooling capacity.

### SUMMARY OF THE INVENTION

The present disclosure is directed to a cordless cryosurgical applicator system. A cordless cryosurgical applicator system can comprise a base unit and one or more rechargeable cryosurgical applicators. The base unit can be use to charge the applicator with a cryogenic fluid such that a cryosurgical procedure can be performed. Following use of the applicator, the applicator can be recharged by attaching the applicator to the base unit and refilling a cryogenic reservoir in the applicator from a cryogenic storage tank contained within the base unit. In some embodiments, the base unit can include a self-contained assembly for generating a cryogenic fluid such that the cordless cryosurgical applicator system is essentially self-sufficient when attached to an electrical power source.

In one aspect of the present disclosure, a cryosurgical applicator system and related method provides a base unit that charges rechargeable applicators with liquid nitrogen (LN2) generated by a self-contained on-board air liquefaction system. The LN2 can be stored within a cryogenic storage reservoir within the base unit until such time that the applicators are to be used for a cryosurgical treatment. Each applicator can then be filled with LN2 such that an applicator tip can be cooled to suitable cryoablation temperatures.

In another aspect of the present disclosure, a cryosurgical applicator system includes one or more rechargeable applicators for performing a cryosurgical treatment. Each rechargeable applicator can be individually, fluidly connected to a base unit that can fill a cryogenic storage reservoir inside each applicator with a cryogenic fluid such as liquid nitrogen. The liquid nitrogen can then be recirculated through the applicator so as to cool an applicator tip to suitable temperature for conducting cryoablation. The applicator tip can have a variety of tip configurations in order to tailor cryosurgical treatment to specific patient anatomy. The rechargeable applicator can also include a heating element and rechargeable battery that is chargeable by the base unit wherein the applicator tip can be warmed for use in thawing frozen tissue or warming areas that are not to be frozen and to also provide a medical profession an ability to conduct multiple cryoablation cycles. In some embodiments, the rechargeable applicator can include a disposable applicator tip that is meant for replacement following a cryosurgical treatment.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a schematic illustration of an embodiment of a cryosurgical treatment system including a plurality of cordless and rechargeable cryosurgical applicators.

FIG. 2 is a side view of an embodiment of an applicator for use with the cryosurgical treatment system of FIG. 1.

FIG. 3 is a side, section view of an embodiment of an applicator for use with the cryosurgical treatment system of FIG. 1.

FIG. 4 is a side, section view of an embodiment of an applicator for use with the cryosurgical treatment system of FIG. 1.

### DETAILED DESCRIPTION

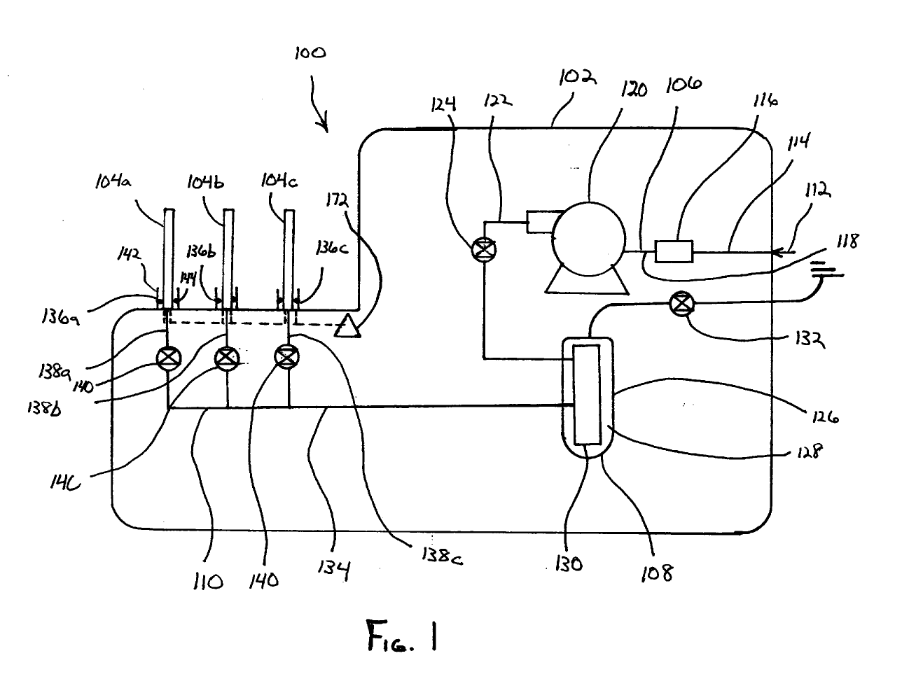

Referring to FIG. 1, a representative embodiment of a cryosurgical applicator system 100 according to the present disclosure can comprise a base unit 102 and one or more applicators herein depicted as applicators 104a, 104b and 104c. As will be understood by one of skill in the art, the cryosurgical applicator system 100 can comprise any number of suitable applicators based primarily upon the types of cryosurgical treatment to be performed and that various features of the base unit 102 can be modified and sized accordingly as will be discussed in further detail below.

Base unit 102 generally comprises a cabinetized or skid-mounted assembly including a cryogen supply portion 106, a cryogen storage tank 108 and a cryogen dispensing portion 110. Cryogen supply portion 106 can comprise any of a variety of suitable technologies and systems for self-contained generation of a cryogenic fluid. As illustrated in FIG. 1, cryogen supply portion 106 can include an ambient air inlet 112, an air supply line 114, an air filter 116, a filtered air line, 118, an air liquefaction assembly 120, a cryogen supply line 122 and a cryogen supply valve 124.

Cryogen storage tank 108 can comprise a suitable storage vessel for storing a volume of cryogen at extremely low temperatures. Typically, cryogen storage tank comprises a vacuum insulated design with an exterior tank 126, a vacuum insulated portion 128 and an inner tank 130. Cryogen storage tank 108 can further comprise a relief valve 132.

Cryogen dispensing portion 110 can comprise a dispensing line 134 and one or more recharging couplers depicted in FIG. 1 as recharging couplers 136a, 136b and 136c. Each of the recharging couplers 136a, 136b, 136c is operably, fluidly coupled to the dispensing line 134 with corresponding coupler lines 138a , 138b and 138c . Each of the coupler lines 138a , 138b , 138c includes an individual check valve 140. Each of the recharging couplers 136a, 136b and 136c also include a receiving member 142 and a sealing member 144 for operably, sealingly receiving the corresponding applicator 104a, 104b and 104c. Sealing member 144 can comprise a variety of sealing members including rubber and rubber-like elastomers formed into suitable sealing configurations including o-rings.

Referring to FIG. 2, a representative embodiment of applicator 104a is illustrated. It will be understood that applicators 104b, 104c can comprise identical and/or similar design with various modifications depending upon the type of cryosurgical treatment to be performed as will be described below. Generally, applicator 104a comprises a handheld body 146 having an application end 148 and a recharging end 150. Application end 148 generally comprises an applicator tip 152 that contacts tissue at a treatment location. The applicator tip 152 can have a variety of configurations based upon the tissue being treating including, for example, a vaginal treatment location, a urethral treatment location, a rectal treatment location, a gastrointestinal treatment location, a nasal treatment location, a bronchial treatment location or an esophageal treatment location. In some embodiments, applicator tip 152 can comprise a disposable tip design such that the tip is disposed of following treatment so as to reduce/eliminate concerns health and safety concerns of using the applicator 104a to treat different individuals. Recharging end 150 is generally dimensioned to snugly fit within the recharging coupler 136a.

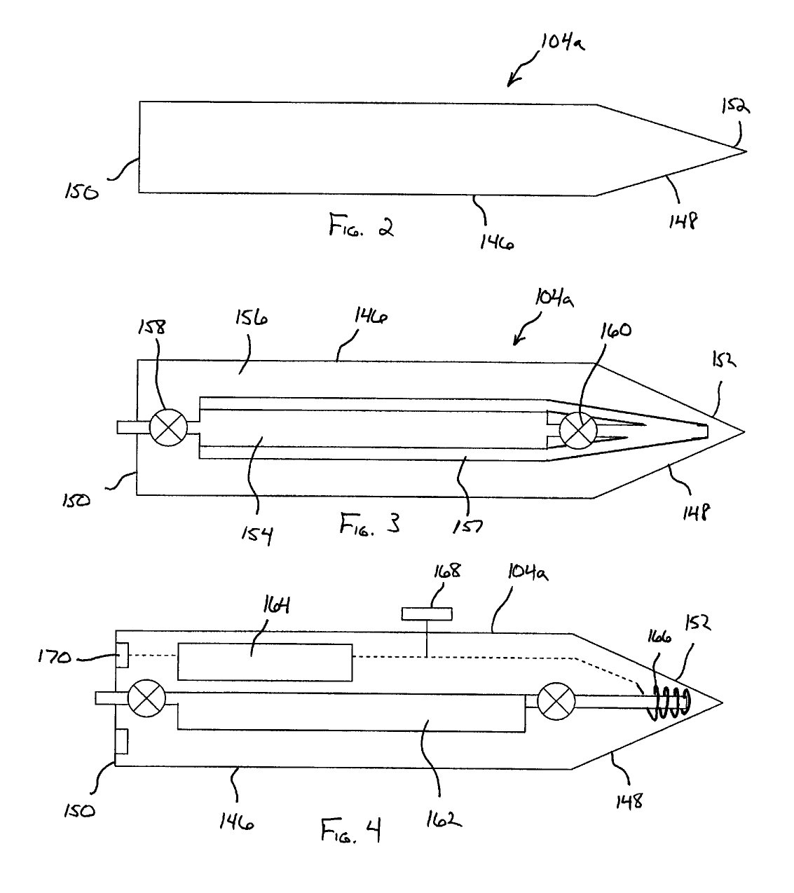

Handheld body 146 can comprise a variety of storage configurations for recharging and utilizing a cryogenic fluid during treatment. As illustrated in FIG. 3, handheld body 146 can include a cylindrical dewer reservoir 154 with an applicator vacuum portion 156 surrounding the reservoir so as to substantially eliminate heat transfer between the reservoir and the ambient environment. The handheld body 154 can further comprise a coaxial return portion 157 so as to further reduce the potential for heat transfer between the cylindrical dewer reservoir 154 and the ambient atmosphere. The cylindrical dewer reservoir 154 can comprise a filling valve 158 that interacts with the recharging coupler 136a so as to recharge and refill the cylindrical dewer reservoir 154 with cryogenic fluid. A dispensing valve 160 can be actuated by a user when the applicator tip 152 is positioned proximate the tissue to be treated. When open, the dispensing valve 160 allows the cryogenic fluid to flow into the applicator tip 152 whereby the cryosurgical procedure can be performed.

Referring to FIG. 4, handheld body 146 in an alternative configuration can comprise a cryogenic storage reservoir 162 and a rechargeable battery 164 operably connected to a resistive heating element 166 in the applicator tip 152. The handheld body 146 can comprise an external control 168 that allows the user to selectively heat or cool the applicator tip 152 such that multiple cryoablation cycles can be performed. The rechargeable battery 164 can be operably interconnected to an electrical contact 170 in the recharging end 150 that provide for an opportunity to simultaneously charge the rechargeable battery 164 and the cryogenic storage reservoir 162 with cryogenic fluid when the applicator 104a is attached to the recharging coupler 136a. Base unit 102 can comprise a power source 172 electrically interconnected to recharging couplers 136a, 136b, 136c so as to interface with the electrical contact 170. Power source 172 can comprise a typical plug assembly for providing power to base unit 102.

In use, the cryosurgical applicator system 100 is able to quickly charge each of the applicators 104a, 104b, 104c due to the self-contained and self-generated cryogenic fluid within cryogen storage tank 108. The cryogenic fluid within the cryogen storage tank 108 can be kept cold through intermittent chilling using the air liquefaction assembly 120. A disposable applicator can be attached to the base unit and chilled before use.

Base unit 102 generates and fills the cryogen storage tank 108 with cryogenic fluid via an air liquefaction process within supply portion 106. Air liquefaction assembly 120 can assume a variety of configurations including, for example, a compact Stirling cryocooler, Gifford-McMahon refrigerator or a membrane based LN2 generator such as, for example, Liquid Nitrogen Plant's available from Cryomech Inc. of Syracuse, N.Y. Using air liquefaction assembly 120, nitrogen is separated from ambient air and is converted from a gaseous form to a liquid form as liquid nitrogen (LN2). The LN2 is then stored within the cryogen storage tank 108.

During a cryosurgical treatment, a medical professional operably positions the applicators 104a, 104b, 104c within the recharging couplers 136a, 136b, 136c. LN2 from the cryogen storage tank 108 flows through each of the coupler lines 138a, 138b, 138c such that the cryogen storage reservoir such as, for example, the cylindrical dewer reservoir 154 or the cryogenic storage reservoir 162 is filled with LN2. When the cryogen storage reservoir is filled, the user can removed the desired applicator from its recharging coupler for use in the desired cryosurgical treatment. As presently contemplated, each of the applicators 104a, 104b, 104c can have a cooling capacity of 25-30 watts when fully charged with LN2. Once the applicator tip 152 has been positioned proximate the tissue to be treated, the user can activate the dispensing valve 160 such that the LN2 within the cryogenic reservoir is circulated through the applicator tip 152 to cool it.

Advantages of using air liquefaction to generate a cryogenic fluid include the use of lower, and therefore safer, pressures. The cryosurgical applicator system 100 is also self-sufficient because it uses air as the supply gas, so it does not need recharging or cylinder replacement. As the base unit 102 can charge the applicators 104a, 104b, 104c without connecting to an external cryogenic fluid supply, cryosurgical applicator system 100 is essentially a cordless system with the exception of an electrical power cord. IN some embodiments, base unit 102 can charge applicators 104a, 104b, 104c through a combination of air filtration and nitrogen liquefaction.

As an alternative to generating a cryogenic fluid using air liquefaction, an embodiment of the cryosurgical applicator system can detachably connectable to a console containing a chilled cryogenic fluid, which can be pumped to the base unit 102 and stored within the cryogen storage tank 108 until it is used to fill the applicators 104a, 104b, 104c. Once the cryogenic fluid has been pumped from the console to the base unit, the base unit can be detached from the console so that cryosurgery can be performed in a cordless manner.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A cordless cryosurgical applicator system comprising:
a base unit including a cryogen supply portion configured to generate a cryogenic fluid, a cryogen storage tank for storing the cryogenic fluid, and a cryogen dispensing portion for dispensing the cryogenic fluid; and
one or more applicators having a handheld body defining an application end and a recharging end, wherein the recharging end is adapted to sealingly engage the cryogen dispensing portion such that a storage reservoir in the handheld body can be filled with cryogenic fluid and wherein the application end includes an applicator tip configured for placement proximate tissue to be treated, wherein a dispensing valve releases the cryogenic fluid from the storage reservoir such that the cryogenic fluid is circulated through the applicator tip.

2. The system of claim 1, wherein the cryogen supply portion includes an air liquefaction assembly for generating liquid nitrogen, the air liquefaction assembly selected from the group consisting of: a compact Stirling cryocooler, a Gifford-McMahon refrigerator and a membrane based liquid nitrogen generator.

3. The system of claim 1, wherein the cryogen dispensing portion includes a dispensing line fluidly interconnecting the cryogen storage tank to at least one recharging coupler, each recharging coupler including a receiving member and a sealing member for operably, sealingly receiving the recharging end the one or more applicators.

4. The system of claim 3, wherein the storage reservoir is a dewar reservoir that includes a filling valve that interacts with the recharging coupler to fill the dewar reservoir when the applicator is attached to the receiving member, the applicator further including a dispensing valve configured to be actuated by a user to allow the cryogenic fluid to circulate through the applicator tip.

5. The system of claim 3, wherein each applicator includes a battery operably connected to a resistive heating element located in the applicator tip.

6. The system of claim 5, wherein each applicator further includes an external control configured to allow a user to selectively heat or cool the applicator tip by either releasing the cryogenic fluid from the storage reservoir into the tip or activating the resistive heating element.

7. The system of claim 5, wherein the battery comprises a rechargeable battery and wherein each applicator further includes an electrical contact in the recharging end that interfaces with a power supply in the base unit when each applicator is attached to the at least one recharging coupler.

8. A cordless disposable applicator for a cryosurgical applicator system, comprising:
a body having an application end and a recharging end;
a storage reservoir within the body for storing a cryogenic fluid received through the recharging end from a base unit; and
an applicator tip located on the application end, the applicator tip configured to perform a cryosurgical treatment when the cryogenic fluid is released from the storage reservoir and circulated through the applicator tip.

9. The system of claim 8, wherein the storage reservoir is a dewar reservoir.

10. The system of claim 8, wherein the body includes a dispensing valve for acuation by a user to allow the cryogenic fluid to flow from the storage reservoir and into the applicator tip.

11. The system of claim 8, wherein the applicator includes a rechargeable battery operably connected to a resistive heating element located in the applicator tip.

12. The system of claim 11, wherein the applicator includes an external control configured to allow a user to selectively heat or cool the applicator tip by either releasing the cryogenic fluid from the storage reservoir into the tip or activating the resistive heating element.

13. The system of claim 12, wherein the applicator further includes an electrical contact in the recharging end that allows the battery to be recharged simultaneously as the storage reservoir is filled with the cryogenic fluid from the base unit.

14. A method of performing a cryosurgical procedure, comprising:
providing a cryosurgical system having a base unit and one or more handheld applicators;
coupling the one or more handheld applicator to one or more recharging couplers on the cryosurgical system;
filling a reservoir in the one or more handheld applicator with a cryogenic fluid retained in a cryogen storage tank in the base unit; and
cooling an applicator tip of the one or more applicators by circulating the cryogenic fluid from the reservoir through the tip.

15. The method of claim 14, further comprising:
generating a cryogenic fluid with an air liquefaction assembly in the base unit; and
storing the cryogenic fluid in a cryogen storage tank.

16. The method of claim 14, further wherein cooling the tip of the one or more applicators comprises activating a dispensing valve in the storage reservoir to circulate the cryogenic fluid through the applicator tip

17. The method of claim 14, further comprising:
freezing the tissue with the applicator tip.

18. The method of claim 1, further comprising:
activating a resistive heating element within the applicator tip; and
thawing the tissue with the applicator tip.

19. The method of claim 18, further comprising:
recharging a battery powering the resistive heating element by placing the one or more handheld applicators in the one or more recharging couplers.

20. The method of claim 14, further comprising:
insulating the reservoir to prevent heat transfer between ambient air and the reservoir.
